(19) United States
(12) Patent Application Publication
(10) Pub. No.: US 2015/0119696 A1
(43) Pub. Date: Apr. 30, 2015

(54) MAGNETIC RESONANCE APPARATUS AND METHOD FOR OPERATION THEREOF

(71) Applicant: Siemens Aktiengesellschaft, Muenchen (DE)

(72) Inventor: Andre de Oliveira, Uttenreuth (DE)

(73) Assignee: Siemens Aktiengesellschaft, Muenchen (DE)

(21) Appl. No.: 14/528,121

(22) Filed: Oct. 30, 2014

(30) Foreign Application Priority Data
Oct. 30, 2013 (DE) 102013222103.7

(51) Int. Cl. A61B 5/00; A61B 5/08; A61B 5/055

(52) U.S. Cl. CPC A61B 5/7285; A61B 5/055; A61B 5/0816; A61B 5/6831
USPC 600/413

(57) ABSTRACT

In a method to operate a magnetic resonance device to acquire magnetic resonance data of an acquisition region of a patient that is moving due to respiration, a breath hold command is automatically given to the patient by an output unit of the magnetic resonance apparatus before acquisition of the magnetic resonance data, and the acquisition of the magnetic resonance data is begun automatically given a trigger signal determined from measurement data of the patient, the trigger signal indicating that breath is being held.

### BACKGROUND OF THE INVENTION

1. Field of the Invention

The invention concerns a method to operate a magnetic resonance apparatus to acquire magnetic resonance data from an acquisition region of a patient that moves during breathing (respiration), of the type wherein before acquisition of the magnetic resonance data, a breath-hold command is given to the patient automatically via an output unit of the magnetic resonance device. The invention also concerns a magnetic resonance apparatus that operates in such a manner.

2. Description of the Prior Art

The acquisition of a magnetic resonance data from acquisition regions of a patient that are moving due to breathing can lead to movement artifacts in magnetic resonance images reconstructed from the magnetic resonance data, particularly for magnetic resonance sequences that have a longer duration. This primarily applies to the acquisition of magnetic resonance data in the chest region or abdominal region of a patient. Various methods have already been proposed in order to reduce or to remedy movement artifacts in images of the chest region and/or abdominal region of a patient due to breathing movement.

For example, it is known to have an acoustic breath-hold command provided by an operator of the magnetic resonance device. Before the measurement (data acquisition), an operator of the magnetic resonance device thereby gives the patient the instructions pertaining to breathing. The operator has complete control over the magnetic resonance apparatus and starts the actual measurement of the magnetic resonance data only when the operator has visual confirmation that the ribcage of the patient is no longer moving. A disadvantage of this method is that the complete attention of the operator of the magnetic resonance device is necessary, which could otherwise be devoted to the checking of other patient-related parameters, or even already to the preparation of the next patient.

The methods for breath triggering or breath gating also have been proposed. Breath-triggered sequences are modified so that, as they begin, a wait is implemented for the occurrence of phases of the breathing cycle in which only a slight movement is present. Various possibilities are known to acquire the trigger signals that indicate reaching the beginning of such a phase of the breathing cycle. Such phases are used repeatedly in breath gating. For example, a discussion of breath triggering and breath gating is found in an article by C. E. Lewis et al., “Comparison of Respiratory Triggering and Gating Techniques for the Removal of Respiratory Artifacts in MR-Imaging”, Radiology 160 (3), Pages 803-810, 1986. Various possibilities have been proposed to acquire the measurement data from which the trigger signals indicating the beginning of the phase in the breathing cycle are determined, for example the acquisition of navigator data with the magnetic resonance device itself. Dedicated breathing measurement devices (in particular breathing belts) are also already known. Breathing belts generally operate with a pneumatic measurement method. A disadvantage of these variants is that, for example, longer measurement times in order to complete the entire exposures are often necessary, in spite of the fact that less patient cooperation is required.

The use of automatic speech commands has also recently been proposed, as disclosed in US 2009/0048505 A1, for example. In such a method, it is no longer necessary of the operator of the magnetic resonance device to personally give the speech commands to the patient; rather, an output unit is provided that emits the speech commands (for example breath hold commands). It is known for a recorded speech message to be played back and emitted as a breath hold command, and the measurement is begun immediately after the end of the breath hold command. However, it is problematic that some patients require a certain amount of time in order to reach the point in time at which the breathing process is actually stopped. Artifacts can thus still occur. The insertion of a pause after the breath hold command and before the beginning of the acquisition of the magnetic resonance data would have the advantage that different pauses would be required for different patients.

### SUMMARY OF THE INVENTION

An object of the invention is to provide a method to acquire magnetic resonance data from a respirating subject that is improved compared to the known techniques, and that also reduces artifacts due to breathing movements for automatically provided breath hold commands.

This object is achieved by a method of the aforementioned type wherein according to the invention, the acquisition of the magnetic resonance data is begun automatically by a trigger signal determined from measurement data of the patient, the trigger signal indicating the halting of breathing.

Only if the trigger signal (which indicates that the breathing has actually been halted by the patient) is present is the acquisition of magnetic resonance data begun; for example, a magnetic resonance sequence is thus started. Through a measurement, the method according to the invention thus ensures that a breath hold has also actually taken place. In this way, the robustness of magnetic resonance acquisitions is improved with automatic speech commands (here breath hold commands) by a trigger signal based on measurement data being used in order to determine the point in time at which the patient actually holds his or her breath. In this way, the image quality is also increased given use of automatic breath hold commands. Overall, the trustworthiness of apparatus customers in the use of automatic speech commands can also be increased.

It is noted that the breath hold command can naturally be part of a sequence of speech commands that are played back via the output unit. For example, it is conceivable to initially give the instruction to take a deep breath, then to exhale, to inhale again and to hold the breath. Only the last command is the actual breath hold command; after this, according to the method the measurement begins only when it is clear from the measurement data that breath is actually held.

According to the invention, it is preferred for the breath hold-indicating measurement data are obtained at least in part from a navigator measurement with the magnetic resonance device. Navigator measurements, in particular one-dimensional navigator measurements that can monitor the position of the diagram, have proven to be an excellent indicator for tracking the breathing movement, such that they can also be used in order to be able to establish the lack of a presence of a breathing movement. Concrete possibilities to realize such a navigator measurement are already widely known in the prior art and do not need to be explained here.

However, it is alternatively or additionally possible for the measurement data to be obtained at least in part from a breathing measurement device, in particular from a breathing belt and/or a breathing measurement device having a pickup coil. Such (possibly less precise) measurement devices can likewise be used within the scope of the present invention, since it only needs the establishment of a halting of the breathing but not an exact determination of a point in time in the breathing cycle. In a breathing measurement device pickup coils can be arranged on the patient, for example, and, due to their movement due to breathing the patient in the magnetic field of the magnetic resonance device, generate induced electrical signals as measurement data.

Measurement data describing a position in the breathing cycle are preferably acquired by the trigger signal being provided depending on an evaluation of the time curve of the measurement data. After the end of the speech command, a cyclical acquisition of measurement data consequently takes place from which a detection can be made as to the position in the breathing cycle in which the patient is presently located. The time curve of these measurement data consequently indicates whether a breath-hold process is present when the position in the breathing cycle that they describe no longer changes, or changes only slightly. Various embodiments to generate the trigger signal based on such cyclically acquired measurement data are accordingly conceivable.

In a simple embodiment the trigger signal is emitted when an absolute value of the difference between the measurement data of the two last measured points in time is smaller than a predetermined threshold. The currently acquired position in the breathing cycle is thus compared with the previously acquired position in the breathing cycle, whereupon the trigger signal is emitted and the acquisition of the magnetic resonance data is started when no movement (or a movement below the predetermined threshold) is present.

It is also conceivable to evaluate the curve over a longer time period, for example so that the trigger signal is emitted when a curve of the at least three last acquired measurement data converges toward a convergence value. However, other possibilities are conceivable to analyze the time curve in order to ensure that a halt of the breathing is actually present.

In addition to the method, the invention also concerns a magnetic resonance apparatus having an output unit for emitting breath hold commands and a control device that is designed to implement the method according to the invention. For example, the control device can include a trigger unit that evaluates measurement data of the patient to generate a trigger signal indicating the halt of the breathing. When the trigger signal is present (emitted), the acquisition of the magnetic resonance data is started. All embodiments regarding the method according to the invention apply analogously to the magnetic resonance apparatus according to the invention, so that the advantages of the method can also be achieved with the apparatus.

### BRIEF DESCRIPTION OF THE DRAWINGS

FIG. 1 is a flowchart of an embodiment of the method according to the invention.

FIG. 2 shows an example of the course of the position in the breathing cycle before the acquisition of magnetic resonance data.

FIG. 3 schematically illustrates a magnetic resonance apparatus according to the invention.

### DESCRIPTION OF THE PREFERRED EMBODIMENTS

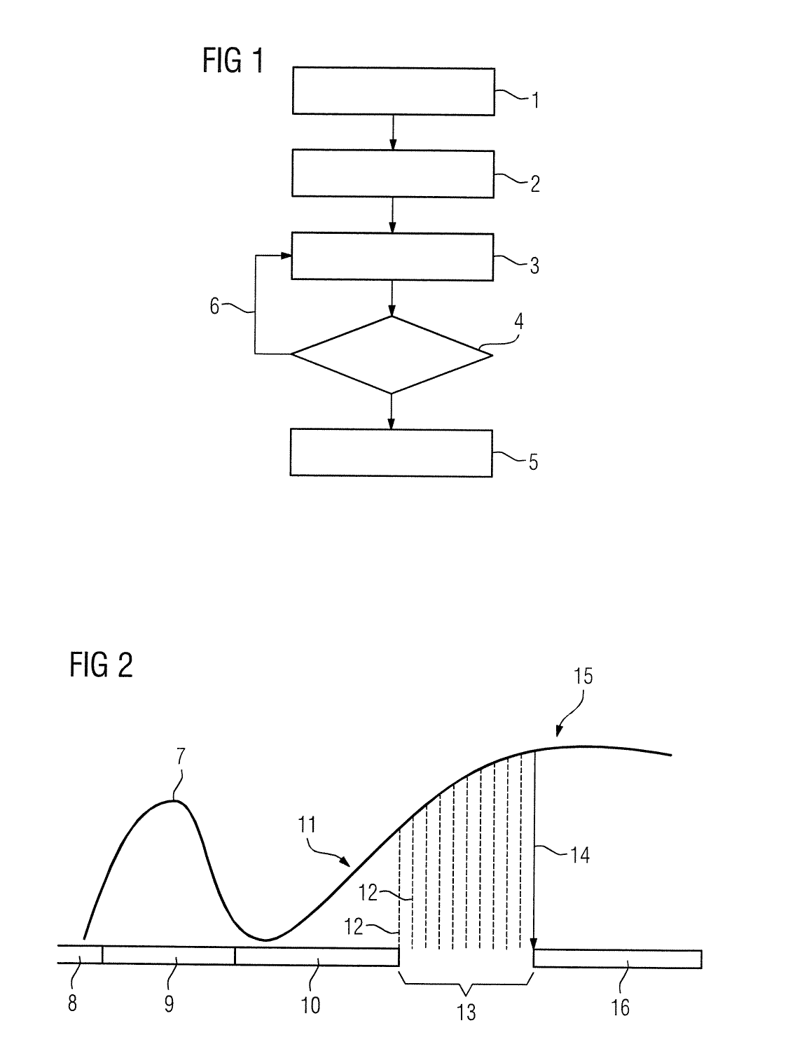

FIG. 1 shows a flowchart of an exemplary embodiment of the method according to the invention in which magnetic resonance data of a patient should be acquired, from the chest region and/or abdomen region as an acquisition region. For this purpose, a breath hold command is typically given to the patient so that the acquisition of the magnetic resonance data can take place during the breath-hold by means of a magnetic resonance sequence and said acquisition is as free of movement artifacts as possible. The typical prepared steps—for example the positioning of the patient within the magnetic resonance device, the selection and parameterization of the measurement program and the like—take place before the implementation of the method as shown in FIG. 1.

In step 1, an output unit of the magnetic resonance device is then used in order to output a series of speech commands wholly automatically, embedded into the workflow of the measurement program, which series is concluded via the breath hold command.

However, the acquisition of the magnetic resonance data is not then started immediately; rather, first in step 2 measurement data are acquired that describe the current breathing position of the patient in the breathing cycle, presently by means of a navigator measurement via the magnetic resonance device. Additionally or alternatively, data about a breathing measurement device—in particular a breathing belt and/or a breathing measurement device comprising a pickup coil—can also be used. The acquisition of the measurement data thereby takes place cyclically, meaning in fixed time intervals.

As soon as measurement data are present at at least two points in time, the difference of the positions in the breathing cycle is considered. If it is less than a predetermined threshold (or even zero), in a step 3 a trigger signal is output according to arrow 4 and the acquisition of the magnetic resonance data begins (step 5). Otherwise, according to arrow 6 additional measurement data are acquired until the condition to generate the trigger signal is present.

It is noted that the evaluation of the time curve of the measurement data can also relate to more than two points in time; for example the convergence at a convergence value can be checked in order to still establish with certainty that the patient holds his or her breath.

This is explained in detail in FIG. 2, which shows an example curve 7 of the breathing position in the breathing cycle given a magnetic resonance examination according to the invention. Speech commands 8, 9, 10 are initially (see step 1) emitted acoustically via the output unit of the magnetic resonance device, wherein the speech command 10 corresponds to the breath hold command. For example, the speech command 8 can be “deeply inhale”, the speech command 9 can be “exhale”, and the speech command 10 serving as a breath hold command can be “inhale and hold breath”. The curve 7 clearly initially shows the inhalation and exhalation, whereupon the rise 11 preceding the breath hold follows given a new inhalation. After the end of the speech command 10, the acquisition of the measurement data (step 2) begins, wherein the dashed lines 12 indicate the cyclical acquisition of measurement data. The acquisition of the measurement data takes place over a time period 13 until the trigger condition is satisfied and the trigger signal is provided (see arrow 14), thus until the breathing position barely changes any more (plateau 15), which indicates that the patient holds his or her breath. The acquisition of the magnetic resonance data can then occur (step 5); see also block 16.

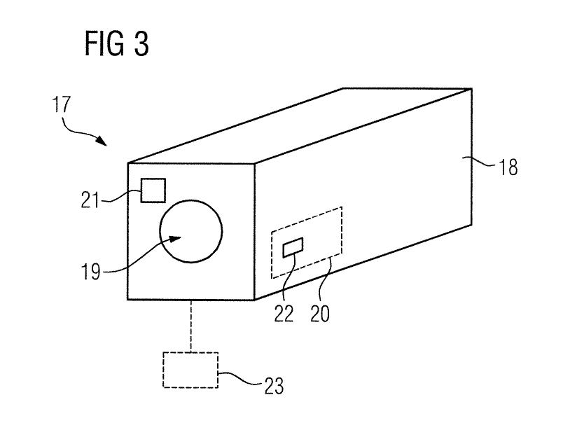

FIG. 3 shows a magnetic resonance apparatus 17 according to the invention. As is known in principle, this apparatus has a basic magnet unit 18 that defines a patient receptacle 19 into which a patient can be driven by a patient bed (not shown). A radio-frequency coil arrangement and a gradient coil arrangement are typically arranged surrounding the patient receptacle 19.

The operation of the magnetic resonance device 17 is controlled by a control device 20 that is also designed to implement the method according to the invention. For this purpose, the control device 20 controls an acoustic output unit 21 in cause to output speech commands to be emitted, in particular the breath hold command. The control device 20 also has a trigger unit 22 that receives and evaluates the measurement data of the navigator measurement, and generates the trigger signal upon arrival of the trigger condition indicating that the patient holds his breath, such that the acquisition of the magnetic resonance data can start. For this purpose, the trigger unit 22 also receives measurement data of an (optionally provided) breathing measurement device 23, for example a breathing belt.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method to operate a magnetic resonance apparatus comprising;
from a computerized control unit, automatically providing a breath hold command to a respirating patient situated in a magnetic resonance apparatus via a command output unit of the magnetic resonance apparatus, before operating said magnetic resonance apparatus to acquire magnetic resonance data from the patient;
acquiring measurement data from the examination subject representing physiological movement of the patient due to the respiration and automatically analyzing said measurement data in said control unit to identify a time at which the patient, after said breath hold command, is physiologically implementing a breath hold; and
at said time, automatically emitting a trigger signal from said control unit that initiates the acquisition of magnetic resonance data from the patient.

2. A method as claimed in claim 1 comprising acquiring said measurement data as a navigator signal by operation of said magnetic resonance apparatus.

3. A method as claimed in claim 1 comprising obtaining said measurement data from a breath measurement device worn by said patient.

4. A method as claimed in claim 3 comprising selecting said breathing measurement device from the group consisting of a breathing belt and a breathing measurement device comprising a pick up coil.

5. A method as claimed in claim 1 comprising acquiring said measurement data as a time curve of a breathing cycle of the patient, and generating said trigger signal dependent on a cyclically reoccurring position in said breathing cycle.

6. A method as claimed in claim 5 comprising generating said trigger signal when an absolute value of a difference between measurement data representing at least two points in time in said breathing cycle is less than a predetermined threshold.

7. A method as claimed in claim 5 comprising generating said trigger signal when a curve of at least three points represented in said measurement data converges on a convergence value.

8. A magnetic resonance apparatus comprising:
a magnetic resonance data acquisition unit;
a computerized control unit configured to automatically provide a breath hold command to a respirating patient situated in the magnetic resonance data acquisition unit via a command output unit of the magnetic resonance data acquisition unit, before operating said magnetic resonance data acquisition unit to acquire magnetic resonance data from the patient;
said control unit being configured to acquire measurement data from the examination subject representing physiological movement of the patient due to the respiration and to automatically analyze said measurement data to identify a time at which the patient, after said breath hold command, is physiologically implementing a breath hold; and
said control unit being configured, at said time, to automatically emit a trigger signal to said magnetic resonance data acquisition unit that initiates the acquisition of magnetic resonance data from the patient.
